(19) United States
(12) Patent Application Publication
(10) Pub. No.: US 2008/0178898 A1
(43) Pub. Date: Jul. 31, 2008

(54) Soap-free shaving foam based on N-acylsarcosinate and free linear saturated fatty acid and shaving process thereof

(76) Inventors: Lionnel Aubert, Asnieres Sur Oise (FR); Lydia Dussault, St Nom le Breteche (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/007,922

(22) Filed: Jan. 17, 2008

Related U.S. Application Data
(60) Provisional application No. 60/897,826, filed on Jan. 29, 2007.

(30) Foreign Application Priority Data
Jan. 17, 2007 (FR) 0752725

(51) Int. Cl. A61K 8/02; A45D 27/00; A61Q 9/02

(52) U.S. Cl. 132/200; 424/47

(57) ABSTRACT

The disclosure relates to soap-free shaving compositions, packaged in a monoblock aerosol device and producing a foam at the outlet of the device, comprising, in a cosmetically acceptable medium:
a) at least one aqueous phase;
b) at least one N-acylsarcosine wherein the acyl radical is C₁₀-C₂₀ and which is from 50% to 90% neutralized;
c) at least one non-neutralized saturated linear C₁₄-C₁₈ fatty acid;
d) at least one nonionic surfactant; and
e) at least one propellant.

The disclosure also relates to a shaving process comprising applying to the surface of skin to be shaved a composition as defined above, followed by shaving the hairs using a razor.

The disclosure also relates to shaving kits comprising:
a) at least one composition as defined above,
b) at least one razor, for example a disposable razor, and/or
c) a means for spreading a shaving composition.

This application claims benefit of U.S. Provisional Application No. 60/897,826, filed Jan. 29, 2007, the contents of which are incorporated herein by reference. This application also claims benefit of priority under 35 U.S.C. §119 to French Patent Application No. FR 0752725, filed Jan. 17, 2007, the contents of which are also incorporated herein by reference.

The present disclosure relates to a soap-free shaving composition packaged in a monoblock aerosol device and producing a foam, comprising at least one N-acylsarcosine, the acyl radical of which is C₁₀-C₂₀ and is from 50% to 90% neutralized; at least one non-neutralized saturated linear C₁₄-C₁₈ fatty acid; at least one nonionic surfactant; and at least one propellant.

The present disclosure also relates to a shaving process that comprises applying to the skin a composition as defined above.

Shaving foams and self-foaming shaving gels are both present in the aerosol shaving products market.

Self-foaming shaving gels are well known and have been described, for example, in U.S. Pat. Nos. 2,995,521, 3,541,581, 4,405,489, 4,528,111, 4,651,503, 5,248,295, 5,308,643, and 5,326,556 and International Patent Application WO 91/07943. Such formulations are in the form of an oil-in-water emulsion in which the self-foaming agent, generally a volatile aliphatic hydrocarbon (i.e., a hydrocarbon of low boiling point) is dissolved in the oily phase and the aqueous phase comprises a water-soluble soap. The product is generally packaged in an aerosol container with a separation such as a piston or a flexible bag to separate the self-foaming agents from the propellant required to expel the product. The product is generally applied in the form of a transparent, translucent or opaque gel that is substantially free of foam until spread onto the skin, at which point the foam is produced by evaporation of the volatile hydrocarbon foaming agent.

Shaving foams are generally in the form of an oil-in-water emulsion wherein the aqueous phase contains a water-soluble soap. The product is generally packaged in a monoblock aerosol container in which the propellant is in mixture with the fluid; the foam becomes formed on leaving the aerosol device. Conventional shaving foam with soap may dry out or increase the roughness of the skin (due to the presence of soap in the formula). To attenuate this effect, shaving foams are typically formulated by incorporating softeners, for example humectants, emollients or silicones. The incorporation of these products, however, may have an influence on the aesthetic quality of the product and may also cause dryness of the skin by repeated use.

For these reasons, “soap-free” shaving products that dry out the skin less were developed with compositions containing N-acylsarcosinates. These starting materials used in their neutralized form (neutralized with sodium hydroxide or TEA) are known for use in aerosol shaving products (Harry's Cosmeticology 7th edition, 1982). However, this type of “soap-free” formulation has very insufficient foam qualities on application.

It would thus be desirable to find novel soap-free shaving foam formulations whose foam qualities are substantially improved, and which do not have one or more of the drawbacks mentioned above.

N-Acylsarcosinates are well-known anionic surfactants of formula:

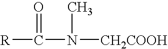

wherein R is a fatty acid hydrocarbon-based chain. These surfactants are generally used in the form of water-soluble salts formed by neutralization with sodium hydroxide, potassium hydroxide, aqueous ammonia or triethanolamine and have been used in a wide range of products, such as shampoos, detergents, toothpastes, shaving creams or hand soaps. For example, aerosol shaving creams containing sarcosinates are described in U.S. Pat. Nos. 3,959,160, 4,113,643 and 4,140,648 and also in Harry's Cosmeticology (7th edition, 1982, page 169—see Croda Cosmetic and Pharmaceutical Formulary Supplement, formula SV11). A non-aerosol shaving cream that may contain an N-acylsarcosinate was described in U.S. Pat. No. 4,892,729 and a non-aerosol shaving gel containing a soap and a sarcosinate was described in U.S. Pat. No. 5,340,571.

The present inventors have discovered, surprisingly and unexpectedly, that it is possible to obtain soap-free shaving foams that produce, on leaving the aerosol, a foam of satisfactory rigidity, without one or more the drawbacks mentioned above, by using an N-acylsarcosine which is from 50% to 90% neutralized, at least one non-neutralized saturated linear C₁₄-C₁₈ fatty acid, and at least one nonionic surfactant.

The present disclosure thus relates to soap-free shaving compositions packaged in a monoblock aerosol device and producing a foam at the outlet of the device, the compositions comprising, in a cosmetically acceptable medium:
a) at least one aqueous phase;
b) at least one N-acylsarcosine, the acyl radical of which is C₁₀-C₂₀ and which is from 50% to 90% neutralized;
c) at least one non-neutralized saturated linear C₁₄-C₁₈ fatty acid;
d) at least one nonionic surfactant; and
e) at least one propellant.

The present disclosure relates to shaving processes that comprise applying to the skin a composition as defined above.

In a further aspect, the present disclosure also relates to shaving kits comprising:
a) at least one composition as defined above,
b) at least one razor, for example a disposable razor, and/or
c) a means for spreading a shaving composition.

As used herein, the term “soap-free” is understood as meaning containing less than 1% by weight of soap relative to the total weight of the composition.

As used herein, the term “non-neutralized fatty acid” means any fatty acid in totally free form.

As used herein, the term “monoblock aerosol device” means a pressurized device with a single compartment into which at least one propellant is introduced with the fluid constituting the shaving composition.

As used herein, the expression “cosmetically acceptable medium” means a medium that is compatible with the skin and/or its integuments, which has a pleasant color, odor and feel and which does not cause any unacceptable discomfort (e.g. stinging, tautness or redness) liable to put a consumer off from using this composition.

The N-acylsarcosines in accordance with the present disclosure are, in at least one embodiment, chosen from those bearing a C₁₂-C₁₈ acyl radical. In a further embodiment, they are chosen from stearoyl sarcosine, myristoyl sarcosine, oleoyl sarcosine, lauroyl sarcosine and cocoyl sarcosine, and mixtures thereof. In yet a further embodiment, they are chosen from stearoyl sarcosine and myristoyl sarcosine, and mixtures thereof. The at least one N-acylsarcosine may be present in an amount ranging from 2% to 15% by weight and, in at least one embodiment, ranging from 4% to 10% by weight relative to the total weight of the composition.

The at least one N-acylsarcosine in accordance with the present disclosure is from 50% to 90% neutralized with a mineral base and/or an organic base. The base may be chosen from mineral bases, for instance potassium hydroxide, sodium hydroxide and aqueous ammonia. It may be chosen from organic bases, for example alkanolamines such as isopropanolamine, monoethanolamine, diethanolamine, triethanolamine, aminoethylpropanol and aminomethylpropanol. In at least one embodiment, the organic base is triethanolamine. The amount of base used depends on the amount of N-acylsarcosine present in the composition.

A sufficient amount of base must be used to neutralize the at least one N-acylsarcosine in the aqueous phase and to produce a pH from 4 to 8.5 and, in at least one embodiment, from 5 to 7. To achieve this pH range, the at least one N-acylsarcosine is, in at least one embodiment, from 60% to 80% neutralized. In at least one embodiment, the at least one N-acylsarcosine will be used in a slight molar access relative to the base. The base is, in at least one embodiment, present in an amount ranging from 1% to 6% relative to the total weight of the composition.

The at least one non-neutralized saturated linear C₁₄-C₈ fatty acid is, in at least one embodiment, chosen from myristic acid, palmitic acid, stearic acid and cetylic acid, and mixtures thereof. They are, in at least one embodiment, present in an amount ranging from 2% to 12% by weight, for example ranging from 4% to 8% by weight, relative to the total weight of the composition.

The at least one aqueous phase of the compositions according to the present disclosure, in at least one embodiment, is present in an amount ranging from 65% to 85% by weight, for example from 70% to 80% by weight, relative to the total weight of the composition.

The nonionic surfactants that may be used according to the present disclosure are well known (see, e.g., in this regard “Handbook of Surfactants” by M. R. Porter, published by Blackie & Son (Glasgow and London), 1991, pp 116-178). Thus, they may be chosen, for example, from fatty alcohols whose fatty chain comprises, in at least one embodiment, from 8 to 20 carbon atoms; polyethoxylated, polypropoxylated or polyglycerolated fatty alcohols, fatty α-diols, fatty alkylphenols or fatty acids, having a fatty chain for example comprising from 8 to 20 carbon atoms, and wherein the number of ethylene oxide or propylene oxide groups for example ranges from 2 to 60 and the number of glycerol groups possibly ranges for example from 2 to 30. Further non-limiting examples include copolymers of ethylene oxide and of propylene oxide, condensates of ethylene oxide and of propylene oxide with fatty alcohols; polyethoxylated fatty amides for example comprising from 2 to 30 mol of ethylene oxide, polyglycerolated fatty amides for example comprising on average from 1 to 5, such as from 1.5 to 4 glycerol groups; polyethoxylated fatty amines for example comprising 2 to 30 mol of ethylene oxide; ethoxylated fatty acid esters of sorbitan for example comprising from 2 to 30 mol of ethylene oxide; fatty acid esters of sucrose, fatty acid esters of polyethylene glycol, (C₆-C₂₄)alkylpolyglycosides, N—(C₆-C₂₄)alkylglucamine derivatives, and amine oxides such as(C₁₀-C₁₄)alkylamine oxides or N—(C₁₀-C₁₄)acylaminopropylmorpholine oxides; and mixtures thereof.

Alkylpolyglucosides that are used in at least one embodiment include those comprising an alkyl group comprising from 6 to 30 carbon atoms such as from 8 to 16 carbon atoms, and containing a hydrophilic group (glucoside) for example comprising from 1.2 to 3 saccharide units. Further non-limiting examples of alkylpolyglucosides include decylglucoside(alkyl-C₉/C₁₁-polyglucoside (1.4)), for instance the product sold under the name Mydol 10® by the company Kao Chemicals, under the name Plantaren 2000 UP® by the company Cognis, and under the name Oramix NS 10® by the company SEPPIC; caprylyl/capryl glucoside, for instance the product sold under the name Oramix CG 110® by the company SEPPIC; laurylglucoside, for instance the products sold under the names Plantaren 1200 N® and Plantacare 1200® by the company Cognis; and cocoglucoside, for instance the product sold under the name Plantacare 818/UP® by the company Cognis.

The maltose derivatives are, for example, those described in European Patent Application No. EP-A-566 438, such as O-octanoyl-6′-D-maltose or O-dodecanoyl-6′-D-maltose described in French Patent Application No. FR-2 739 556.

Among the polyglycerolated fatty alcohols that may be mentioned is polyglycerolated dodecanediol (3.5 mol of glycerol), such as the product manufactured under the name Chimexane NF® by the company Chimex.

The at least one nonionic surfactant, in at least one embodiment, is chosen from:
fatty alcohols wherein the fatty chain is C₈-C₂₀ and for example C₁₂-C₁₈, for instance myristyl alcohol, lauryl alcohol, stearyl alcohol and octyldodecanol;
polyoxyethylenated ethers of fatty alcohols wherein the fatty chain is C₈-C₂₀ and for example C₁₂-C₁₈ and which comprise from 2 to 60 and for example from 2 to 30 ethylene oxide units. Among these compounds, non-limiting examples include Oleth-20, Steareth-21, Ceteth-20, Laureth-4 and Laureth-23. A mixture of Laureth-4 and Laureth-23 will, in at least one embodiment, be chosen.

The at least one nonionic surfactant is, in at least one embodiment, present in an amount ranging from 1% to 20% by weight, for example from 3% to 10% by weight, relative to the total weight of the composition.

The at least one propellant is, in at least one embodiment, chosen from volatile hydrocarbons and volatile halohydrocarbons. The boiling point of the propellant, in at least one embodiment, ranges from −20 to 40° C. The at least one propellant that may be used according to the present disclosure is, in at least one embodiment, chosen from C₄-C₆ aliphatic hydrocarbons such as n-pentane, isopentane, neopentane, n-butane and isobutane, and mixtures thereof. In another embodiment, an isopentane/butane/propane mixture will be used. The propellant is, in at least one embodiment, present in an amount ranging from 1 % to 10% by weight, for example from 2% to 6% by weight, relative to the total weight of the composition.

In at least one embodiment, the compositions also comprise surfactants chosen from anionic surfactants and amphoteric or zwitterionic surfactants, and mixtures thereof.

Non-limiting examples of anionic surfactants in accordance with the present disclosure that may be used include carboxylates, oxyethylenated or non-oxyethylenated alkyl sulfates, sulfonates, alkyl sulfoacetates, phosphates, polypeptides, and anionic derivatives of alkyl polyglucoside, and mixtures thereof.

Non-limiting examples of carboxylates that may be mentioned include:
amido ether carboxylates (AEC), for instance sodium laurylamido ether carboxylate (3 EO) sold under the name Akypo Foam 30® by the company Kao Chemicals;
polyoxyethylenated carboxylic acid salts, for instance oxyethylenated (6 EO) sodium lauryl ether carboxylate (65/25/10 C12-14-16) sold under the name Akypo Soft 45 NV® by the company Kao Chemicals; polyoxyethylenated fatty acids of olive oil and of carboxymethyl, for example sold under the name Olivem 400® by the company Biologia E Technologia; oxyethylenated (6 EO) sodium tridecyl ether carboxylate sold under the name Nikkol ECTD-6NEX® by the company Nikkol;
acetates such as sodium 2-(2-hydroxyalkyloxy)acetate sold under the name Beaulight SHAA by the company Sanyo;
alaninates, for instance the sodium N-lauroyl-N-methylamidopropionate sold under the name Sodium Nikkol Alaninate LN 30® by the company Nikkol or under the name Alanone ALE® by the company Kawaken, and the N-lauroyl N-methylalanine triethanolamine sold under the name Alanone ALTA® by the company Kawaken; acylglutamates, for instance the triethanolamine monococoylglutamate sold under the name Acylglutamate CT-12® by the company Ajinomoto, and the triethanolamine lauroylglutamate sold under the name Acylglutamate LT-12® by the company Ajinomoto; aspartates, for instance the mixture of triethanolamine N-lauroyl aspartate and of triethanolamine N-myristoyl aspartate sold under the name Asparack® by the company Mitsubishi; glycinates, for instance the sodium N-cocoylglycinate sold under the names Amilite GCS-12® and Amilite GCK 12 by the company Ajinomoto;
citrates such as the oxyethylenated (9 EO) citric monoester of coco alcohols sold under the name Witconol EC 1129 by the company Goldschmidt; and
galacturonates, for instance the sodium dodecyl-D-galactoside uronate sold by the company Soliance.

Non-limiting examples of oxyethylenated or non-oxyethylenated alkyl sulfates include the sodium lauryl ether sulfate (70/30 C12-14) (2.2 EO) sold under the name Sipon AOS 225® by the company Cognis, the ammonium lauryl ether sulfate (70/30 C12-14) (3 EO) sold under the name Sipon LEA 370® by the company Cognis, the ammonium (C12-C14) alkyl ether (9 EO) sulfate sold under the name Rhodapex AB/20® by the company Rhodia Chimie, and the mixture of sodium and magnesium lauryl and oleyl ether sulfate sold under the name Empicol BSD 52 by the company Albright & Wilson.

Non-limiting examples of sulfonates include (1) α-olefin sulfonates, for instance the sodium α-olefin sulfonate (C14-16) sold under the name Bio-terge AS-40® by the company Stepan, under the names Witconate AOS Protege® and Sulframine AOS PH 12® by the company Witco, the sodium secondary olefin sulfonate sold under the name Hostapur SAS 30® by the company Clariant; (2) isethionates, for instance sodium cocoyl isethionate, such as the product sold under the name Jordapon CI P® by the company Jordan, (3) taurates, for instance the sodium salt of palm kernel oil methyltaurate sold under the name Hostapon CT Paste® by the company Clariant; N-acyl N-methyltaurates, for instance the sodium N-cocoyl N-methyltaurate sold under the name Hostapon LT-SF® by the company Clariant or sold under the name Nikkol CMT-30-T® by the company Nikkol, and the sodium palmitoyl methyltaurate sold under the name Nikkol PMT® by the company Nikkol.

Non-limiting examples of sulfosuccinates include the oxyethylenated (3 EO) lauryl alcohol monosulfosuccinate (70/30 C12/C14) sold under the names Setacin 103 Special®, Rewopol SB-FA 30 K 4® by the company Witco, the disodium salt of a C12-C14 alcohol hemisulfosuccinate sold under the name Setacin F Special Paste® by the company Zschimmer Schwarz, the oxyethylenated (2 EO) disodium oleamidosulfosuccinate sold under the name Standapol SH 135® by the company Cognis, and the oxyethylenated (5 EO) laurylamide monosulfosuccinate sold under the name Lebon A-5000® by the company Sanyo.

Non-limiting examples of phosphates include monoalkyl phosphates and dialkyl phosphates, such as the lauryl monophosphate sold under the name MAP 20® by the company Kao Chemicals, the potassium salt of dodecylphosphoric acid, as a mixture of monoester and diester (mainly diester) sold under the name Crafol AP-31® by the company Cognis, the mixture of octylphosphoric acid monoester and diester sold under the name Crafol AP-20® by the company Cognis, and the mixture of ethoxylated (7 mole of EO) 2-butyloctanol phosphoric monoester and diester sold under the name Isofol 12 7 EO Phosphate Ester® by the company Condea.

Non-limiting examples of polypeptides (which are compounds obtained by condensing a fatty chain onto cereal, e.g., wheat and oat, amino acids) include the potassium salt of hydrolysed lauroyl wheat protein sold under the name Aminofoam W OR® by the company Croda; the triethanolamine salt of hydrolysed cocoyl soybean protein sold under the name May-Tein SY® by the company Maybrook; the sodium salt of oat lauroyl amino acids sold under the name Proteol Oat® by the company SEPPIC; collagen hydrolysate grafted onto coconut fatty acid, sold under the name Geliderm 3000® by the company Deutsche Gelatine; and soybean proteins acylated with hydrogenated coconut acids, sold under the name Proteol VS 22® by the company SEPPIC.

Non-limiting examples of anionic alkylpolyglucoside derivatives include the glyceryl citrates, tartrates, sulfosuccinates, carbonates and ethers obtained from alkyl polyglucosides. Further non-limiting examples include the sodium salt of cocoylpolyglucoside (1,4) tartaric ester, sold under the name Eucarol AGE-ET® by the company Cesalpinia; the disodium salt of cocoylpolyglucoside (1,4) sulfosuccinic ester, sold under the name Essai 512 MP® by the company SEPPIC; the sodium salt of cocoylpolyglucoside (1,4) citric ester sold under the name Eucarol AGE-EC® by the company Cesalpinia, and the sodium lauryl polyglucoside ether carboxylate sold under the name Plantapon LGC Sorb by the company Cognis.

C₆-C₂₄ alkyl ether sulfate salts comprising from 1 to 30 ethylene oxide groups may, in at least one embodiment, be used, for example the alkali metal or alkaline-earth metal, ammonium, amine or amino alcohol salts and for example the sodium salts such as oxyethylenated sodium (C₁₂-C₁₄)alkyl ether sulfates comprising a mean number of ethylene oxide groups ranging from 1 to 4, and for example sodium laureth sulfate (CTFA name).

Non-limiting examples of amphoteric or zwitterionic surfactants that may be used include alkylbetaines, N-alkylamidobetaines and derivatives thereof, sultaines, alkylpolyaminocarboxylates (APAC) and alkylamphoacetates, and mixtures thereof.

Non-limiting examples of alkylbetaines include cocobetaine, for instance the product sold under the name Dehyton AB-30® by the company Cognis; laurylbetaine, for instance the product sold under the name Genagen KB® by the company Clariant; oxyethylenated (10 EO) laurylbetaine, for instance the product sold under the name Lauryl Ether (10 OE) Betaine® by the company Shin Nihon Rica; and oxyethylenated (10 EO) stearylbetaine, for instance the product sold under the name Stearyl Ether (10 OE) Betaine® by the company Shin Nihon Rica.

Non-limiting examples of N-alkylamidobetaines and derivatives thereof include the cocamidopropylbetaine sold under the name Lebon 2000 HG® by the company Sanyo or under the name Empigen BB® by the company Albright & Wilson; and the lauramidopropylbetaine sold under the name Rewoteric AMB12P® by the company Witco.

A non-limiting example of sultaine is the cocoylamidopropylhydroxy-sulfobetaine sold under the name Crosultaine C-50® by the company Croda.

Non-limiting examples of alkylpolyaminocarboxylates (APAC) include the sodium cocoylpolyaminocarboxylate sold under the name Ampholak 7 CX/C® and Ampholak 7 CX® by the company Akzo Nobel; the sodium stearylpolyaminocarboxylate sold under the name Ampholak 7 TX/C® by the company Akzo Nobel; and the sodium carboxymethyloleylpolypropylamine sold under the name Ampholak XO7/C® by the company Akzo Nobel.

Non-limiting examples of alkylamphoacetates include N-disodium N-cocoyl-N-carboxymethoxyethyl-N-carboxymethylethylenediamine (CTFA name: disodium cocamphodiacetate), for instance the product sold under the name Miranol C2M Concentrate NP® by the company Rhodia Chimie; and N-sodium N-cocoyl-N-hydroxyethyl-N-carboxymethylethylenediamine (CTFA name: sodium cocamphoacetate).

Among the amphoteric surfactants that may be used, in at least one embodiment, are:
cocobetaine such as the commercial products Mirataine BB/FLA from Rhodia or Empigen BB/FL from Huntsman,
lauroybetaine and sodium chloride such as the commercial product Empigen BB/LS from Huntsman, and
disodium cocoamphodiacetate, for instance the product sold under the trade name Miranol® C2M Concentrate by the company Rhodia, and mixtures thereof.

The anionic and/or amphoteric or zwitterionic surfactant(s) is (are), in at least one embodiment, present in an amount ranging from 1% to 10% by weight, for example from 2% to 7% by weight, relative to the total weight of the composition.

The compositions of the present disclosure may also contain at least one water-soluble gelling agent or at least one thickener to improve the consistency and stability of the foam.

Among these gelling agents, non-limiting examples include hydroxyalkylcellulose polymers, for instance hydroxyethylcellulose or hydroxypropylcellulose (products sold, respectively, under the trade name Natrosol or Klucel); copolymers of acrylic acid and of polyallylsucrose (products sold under the trade name Carbopol); carboxymethylcellulose and cellulose methyl ether (products sold under the trade name Methocel); natural or synthetic gums, and starches. In at least one embodiment, the at least one thickener is chosen from hydroxyethylcellulose and hydroxypropylcellulose, and mixtures thereof. The at least one gelling agent or thickener is, in at least one embodiment, present in an amount ranging from 0.01% to 5% by weight, for example from 0.05% to 2% by weight, relative to the total weight of the composition.

The compositions of the present disclosure may also contain at least one short-chain polyol to improve the foam qualities and/or the stability of the composition. The at least one short-chain polyol is, in at least one embodiment, present in an amount less than or equal to 15% by weight, for example ranging from 0.25% to 10% by weight, relative to the total weight of the composition. Non-limiting examples of the at least one short-chain polyol that may be used include glycerol and propylene glycol, and mixtures thereof.

The shaving compositions according to the disclosure may also comprise a variety of standard cosmetic ingredients to improve the aesthetic qualities and the performance of these compositions.

The compositions according to the disclosure may also comprise at least one conditioning cationic polymer to improve the lubricity and feel of the skin after shaving. Non-limiting examples include quaternary ammonium salts of hydroxyethylcellulose, for instance Polyquaternium-10 or Polyquaternium-24.

Further non-limiting examples of the at least one conditioning cationic polymer include:
Polyquaternium 5, such as the product Merquat 5 sold by the company Calgon;
Polyquaternium 6, such as the product Salcare SC 30 sold by the company Ciba, and the product Merquat 100 sold by the company Calgon;
Polyquaternium 7, such as the products Merquat S, Merquat 2200 and Merquat 550 sold by the company Calgon; and the product Salcare SC 10 sold by the company Ciba;
Polyquaternium 11, such as the products Gafquat 755, Gafquat 775N and Gafquat 734 sold by the company ISP;
Polyquaternium 15, such as the product Rohagit KF 720 F sold by the company Rohm;
Polyquaternium 16, such as the products Luviquat FC905, Luviquat FC370, Luviquat HM552 and Luviquat FC550 sold by the company BASF;
Polyquaternium 22, such as the product Merquat 280 sold by the company Calgon;
Polyquaternium 28, such as the product Styleze CC10 sold by the company ISP;
Polyquaternium 39, such as the product Merquat Plus 3330 sold by the company Calgon;
Polyquaternium 44, such as the product Luviquat Care sold by the company BASF;
Polyquaternium 46, such as the product Luviquat Hold sold by the company BASF; and
Polyquaternium 47, such as the product Merquat 2001 sold by the company Calgon.

Further non-limiting examples of the at least one conditioning cationic polymer that may also be used include cationic guars, such as the product Jaguar sold by the company Rhodia.

The at least one conditioning cationic polymer is, in at least one embodiment, present in an amount ranging from 0.05% to 2% by weight, for example ranging from 0.1% to 1% by weight, relative to the total weight of the composition.

Other additives may also be used in the compositions of the disclosure, for instance:
humectants, for instance sorbitol;
emollients such as fatty esters, for instance isopropyl myristate, decyl oleate, 2-ethylhexyl palmitate, PEG-7 glyceryl cocoate and glyceryl linoleate; propoxylated fatty ethers, for instance PPG-10 cetyl ether and PPG-11 stearyl ether; diglycerides or triglycerides, for instance lecithin, the mixture of capric/caprylic triglycerides, PEG-10 soy sterol and plant oils;
refreshing agents and calmatives, for instance menthol, Aloe, allantoin, lanolin, bisabolol and hyaluronic acid;
lubricants, for instance polyethylene glycols (for example PEG-14M and PEG-23M), fluorinated surfactants, silicones (for example dimethicone, dimethiconol, dimethicone copolyol, stearyl dimethicone, cetyl dimethicone copolyol, cyclomethicone);
vitamins, including precursors and derivatives, for instance panthenol, tocopheryl acetate, niacinamide, retinyl palmitate and vitamin A palmitate;
fillers;
dyes;
fragrances;
antioxidants;
antibacterial agents and/or antifungal agents; and
preserving agents (for example methylchloroisothiazolinone, methylisothiazolinone, DMDM hydantoin and iodopropionyl butyl carbamate).

Among the fillers that may be used according to the disclosure, non-limiting examples include organic powders. In the present patent application, the term “organic powder” means any solid that is insoluble in the medium at room temperature (25° C.).

As organic powders that may be used in the composition of the disclosure, non-limiting examples include polyamide particles for example those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; polymethyl methacrylate microspheres, sold under the name Microsphere M-100 by the company Matsumoto or under the name Covabead LH85 by the company Wackherr; ethylene-acrylate copolymer powders, for instance those sold under the name Flobeads by the company Sumitomo Seika Chemicals; expanded powders such as hollow microspheres and for example microspheres formed from a terpolymer of vinylidene chloride, of acrylonitrile and of methacrylate and sold under the name Expancel by the company Kemanord Plast under the references 551 DE 12 (particle size of about 12 μm and density of 40 kg/m³), 551 DE 20 (particle size of about 30 μm and density of 65 kg/m³) and 551 DE 50 (particle size of about 40 μm), or the microspheres sold under the name Micropearl F 80 ED by the company Matsumoto; powders of natural organic materials such as starch powders, for example of corn starch, wheat starch or rice starch, which may or may not be crosslinked, such as the starch powder crosslinked with octenylsuccinate anhydride, sold under the name Dry-Flo by the company National Starch; silicone resin microbeads such as those sold under the name Tospearl by the company Toshiba Silicone, for example Tospearl 240; amino acid powders such as the lauroyllysine powder sold under the name Amihope LL-11 by the company Ajinomoto; particles of wax microdispersion, which for example have mean sizes of less than 1 μm such as ranging from 0.02 μm to 1 μm, and which consist essentially of a wax or a mixture of waxes, such as the products sold under the name Aquacer by the company Byk Cera, and for example Aquacer 520 (mixture of synthetic and natural waxes), Aquacer 514 or 513 (polyethylene wax), Aquacer 511 (polymer wax), or such as the products sold under the name Jonwax 120 by the company Johnson Polymer (mixture of polyethylene wax and paraffin wax) and under the name Ceraflour 961 by the company Byk Cera (micronized modified polyethylene wax); and mixtures thereof.

A person skilled in the art will take care to select the optional additional compound(s) mentioned above and/or the amounts thereof such that the advantageous properties intrinsically associated with the compositions in accordance with the disclosure are not, or are not substantially, adversely affected by the envisaged addition(s).

The shaving compositions of the present disclosure are packaged in a monoblock aerosol device (containing only one compartment) in which the propellant and the fluid are mixed.

The present disclosure also relates to a shaving kit comprising:
a) at least one shaving foam as defined above,
b) at least one razor, for example a disposable razor, and/or
c) a means for spreading a shaving composition.

According to one embodiment of the present disclosure, the kit may also comprise an aftershave composition to soothe razor burn.

The examples that follow serve to illustrate the present disclosure. The amounts indicated are weight percentages, unless otherwise mentioned, and the names of the compounds are the chemical names or the CTFA names (International Cosmetic Ingredient Dictionary and Handbook), depending on the case.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### EXAMPLE

The soap-free shaving foams 1 to 9 below packaged in monoblock aerosols were prepared. The amounts are indicated as weight percentages relative to the total weight of the composition.

| Ingredients | Ex 1 (*) | Ex 2 (*) | Ex 3 (*) | Ex 4 | Ex 5 |
|---|---|---|---|---|---|
| 2,6-Dimethyl-7-octen-2-ol | 0.29 | 0.29 | 0.29 | 0.29 | 0.29 |
| Liquid paraffin | — | 2.87 | — | — | — |
| Lauric acid (C₁₂) | — | — | 2.87 | — | — |
| Myristic acid (C₁₄) | — | — | — | 2.87 | — |
| Palmitic acid (C₁₆) | — | — | — | — | 2.87 |
| Glycerol | 4.80 | 4.80 | 4.80 | 4.80 | 4.80 |
| Polyquaternium-7 (Merquat S) | 0.48 | 0.48 | 0.48 | 0.48 | 0.48 |
| Hydroxyethylcellulose | 1.00 | 1.00 | 1.00 | 1.00 | 1.00 |
| (MW: 1 000 000) | | | | | |
| Isobutane/propane/butane | 4.25 | 4.25 | 4.25 | 4.25 | 4.25 |
| (56/24/20) | | | | | |
| Stearoyl sarcosine (and) | 2.87 | 2.87 | 2.87 | 2.87 | 2.87 |
| myristoyl sarcosine (75%/25%) | | | | | |
| Triethanolamine | 1.19 | 1.19 | 1.19 | 1.19 | 1.19 |
| Dimethiconol stearate (Mirasil | 0.67 | 0.67 | 0.67 | 0.67 | 0.67 |
| Wax-S from Rhodia) | | | | | |
| Steareth-100 | 0.48 | 0.48 | 0.48 | 0.48 | 0.48 |
| Laureth-23 | 4.80 | 4.80 | 4.80 | 4.80 | 4.80 |
| Xanthan gum (Rhodicare CFT | 1.00 | 1.00 | 1.00 | 1.00 | 1.00 |
| from Rhodia) | | | | | |
| Lauryl betaine (and) sodium | 4.80 | 4.80 | 4.80 | 4.80 | 4.80 |
| chloride (Empigen BB/LS from | | | | | |
| Huntsman) | | | | | |
| Preserving agents | qs | qs | qs | qs | qs |
| Water | qs 100 g | qs 100 g | qs 100 g | qs 100 g | qs 100 g |
| Rigidity of the shaving foam | 3 | 3 | 4.2 | 11.5 | 11.5 |
| (in grams) | | | | | |

| Ingredients | Ex 6 | Ex 7 (*) | Ex 8 (*) | Ex 9 (*) |
|---|---|---|---|---|
| 2,6-Dimethyl-7-octen-2-ol | 0.29 | 0.29 | 0.29 | 0.29 |
| Stearic acid (C₁₈) | 2.87 | | | |
| Isostearic acid (branched C₁₈) | — | 2.87 | — | — |
| Oleic acid (unsaturated C₁₈) | — | — | 2.87 | — |
| Behenic acid (C₂₀/C₂₁) | | | | 2.87 |
| Glycerol | 4.80 | 4.80 | 4.80 | 4.80 |
| Polyquaternium-7 (Merquat S) | 0.48 | 0.48 | 0.48 | 0.48 |
| Hydroxyethylcellulose | 1.00 | 1.00 | 1.00 | 1.00 |
| (MW: 1 000 000) | | | | |
| Isobutane/propane/butane (56/24/20) | 4.25 | 4.25 | 4.25 | 4.25 |
| Stearoyl sarcosine (and) myristoyl | 2.87 | 2.87 | 2.87 | 2.87 |
| sarcosine (75%/25%) | | | | |
| Triethanolamine | 1.19 | 1.19 | 1.19 | 1.19 |
| Dimethiconol stearate (Mirasil Wax-S | 0.67 | 0.67 | 0.67 | 0.67 |
| from Rhodia) | | | | |
| Steareth-100 | 0.48 | 0.48 | 0.48 | 0.48 |
| Laureth-23 | 4.80 | 4.80 | 4.80 | 4.80 |
| Xanthan gum (Rhodicare CFT from | 1.00 | 1.00 | 1.00 | 1.00 |
| Rhodia) | | | | |
| Lauryl betaine (and) sodium chloride | 4.80 | 4.80 | 4.80 | 4.80 |
| (Empigen BB/LS from Huntsman) | | | | |
| Preserving agents | qs | qs | qs | qs |
| Water | qs 100 g | qs 100 g | qs 100 g | qs 100 g |
| Rigidity of the shaving foam (in | 12 | 3.6 | 3 | 3.1 |
| grams) | | | | |

(*) outside the invention

The rigidity of each shaving foam was measured at 25° C. using a TA XT2i texture analyser manufactured by the company Thermo, equipped with an SMS P/0-5 HS 0.5-inch diameter hemispherical Delrin cylinder probe. The rigidity (expressed in grams) of each product was measured in compression by the said cylinder at a speed of 2 mm/s over a distance of 25 mm.

It was observed that the liquid paraffin, lauric acid (C₁₂), oleic acid (unsaturated C₁₈), isostearic acid (branched C₁₈ fatty acid) and behenic acid (linear C₂₀/C₂₁ fatty acid) of Examples 2, 7, 8 and 9 led to shaving foams of low rigidity (less than or equal to 3.6). These oils afforded no or virtually no improvement compared with Example 1 not containing any free fatty acid.

It was observed that the saturated linear C₁₄-C₁₈ fatty acids of inventive Examples 4, 5 and 6 led, surprisingly, to foams of higher rigidity (11.5 or 12 g).

What is claimed is:

1. A soap-free shaving composition, packaged in a monoblock aerosol device and producing a foam at the outlet of said device, said composition comprising, in a cosmetically acceptable medium:
a) at least one aqueous phase;
b) at least one N-acylsarcosine in which the acyl radical is C₁₀-C₂₀ and which is from 50% to 90% neutralized;
c) at least one non-neutralized saturated linear C₁₄-C₁₈ fatty acid;
d) at least one nonionic surfactant; and
e) at least one propellant.

2. The composition according to claim 1, wherein the acyl radical of the at least one N-acylsarcosine is C₁₂-C₈.

3. The composition according to claim 1, wherein the at least one N-acylsarcosine is chosen from stearoyl sarcosine, myristoyl sarcosine, oleoyl sarcosine, lauroyl sarcosine and cocoyl sarcosine, and mixtures thereof.

4. The composition according to claim 3, wherein the at least one N-acylsarcosine is chosen from stearoyl sarcosine and myristoyl sarcosine, and mixtures thereof.

5. The composition according to claim 1, wherein the at least one N-acylsarcosine is present in an amount ranging from 2% to 15% by weight relative to the total weight of the composition.

6. The composition according to claim 1, wherein the at least one N-acylsarcosine is from 60% to 80% neutralized.

7. The composition according to claim 1, wherein the at least one aqueous phase is present in an amount ranging from 65% to 85% by weight relative to the total weight of the composition.

8. The composition according to claim 1, wherein the at least one non-neutralized saturated linear C₁₄-C₁₈ fatty acid is chosen from myristic acid, palmitic acid, stearic acid and cetylic acid, and mixtures thereof.

9. The composition according to claim 1, wherein the at least one non-neutralized saturated linear C₁₄-C₁₈ fatty acid is present in an amount ranging from 2% to 12% by weight relative to the total weight of the composition.

10. The composition according to claim 1, wherein the at least one nonionic surfactant is chosen from fatty alcohols; polyethoxylated, polypropoxylated or polyglycerolated fatty alcohols, fatty α-diols, fatty alkylphenols or fatty acids; copolymers of ethylene oxide and of propylene oxide; condensates of ethylene oxide and propylene oxide with fatty alcohols; polyethoxylated fatty amides; polyglycerolated fatty amides; polyethoxylated fatty amines; ethoxylated fatty acid esters of sorbitan; fatty acid esters of sucrose, fatty acid esters of polyethylene glycol; (C₆-C₂₄)alkylpolyglycosides; N—(C₆-C₂₄)alkylglucamine derivatives; amine oxides, and mixtures thereof.

11. The composition according to claim 10, wherein the at least one nonionic surfactant is chosen from:
fatty alcohols comprising a C₈-C₂₀ fatty chain;
polyoxyethylenated ethers of fatty alcohols comprising a C₈-C₂₀ fatty chain and further comprising from 2 to 60 ethylene oxide units; and mixtures thereof.

12. The composition according to claim 1, wherein the at least one nonionic surfactant is present in an amount ranging from 1% to 20% by weight relative to the total weight of the composition.

13. The composition according to claim 1, wherein the at least one propellant is chosen from volatile hydrocarbons and volatile halohydrocarbons.

14. The composition according to claim 13, wherein the at least one propellant is chosen from C₄-C₆ aliphatic hydrocarbons.

15. The composition according to claim 14, wherein the at least one propellant is chosen from n-pentane, isopentane, neopentane, n-butane and isobutane, and mixtures thereof.

16. The composition according to claim 13, wherein the at least one propellant is an isopentane/butane/propane mixture.

17. The composition according to claim 1, wherein the at least one propellant is present in an amount ranging from 1% to 10% by weight relative to the total weight of the composition.

18. The composition according to claim 1, wherein the composition further comprises at least one surfactant chosen from anionic surfactants and amphoteric or zwitterionic surfactants, and mixtures thereof.

19. The composition according to claim 18, wherein the at least one surfactant is chosen from anionic surfactants. chosen from carboxylates, oxyethylenated or non-oxyethylenated alkyl sulfates, sulfonates, alkyl sulfoacetates, phosphates, polypeptides and anionic derivatives of alkylpolyglucoside, and mixtures thereof.

20. The composition according to claim 19, wherein the anionic surfactants are chosen from C₆-C₂₄ alkyl ether sulfate salts comprising from 1 to 30 ethylene oxide groups.

21. The composition according to claim 18, wherein the at least one surfactant is chosen from amphoteric or zwitterionic surfactants chosen from alkylbetaines, N-alkylamidobetaines and derivatives thereof, sultaines, alkyl-polyaminocarboxylates (APAC) and alkylamphoacetates, and mixtures thereof.

22. The composition according to claim 18, wherein the at least one surfactant chosen from anionic surfactants and amphoteric or zwitterionic surfactants, and mixtures thereof is present in an amount ranging from 1% to 10% by weight relative to the total weight of the composition.

23. The composition according to claim 1, wherein the composition further comprises at least one water-soluble gelling agent and/or at least one thickener.

24. The composition according to claim 23, wherein the at least one water-soluble gelling agent and/or the at least one thickener is chosen from hydroxyalkylcellulose polymers; copolymers of acrylic acid and of polyallylsucrose; carboxymethylcellulose and cellulose methyl ether; natural or synthetic gums; and starches.

25. The composition according to claim 23, wherein the at least one water-soluble gelling agent and/or the at least one thickener is present in an amount ranging from 0.05% to 5% by weight relative to the total weight of the composition.

26. The composition according to claim 1, wherein the composition further comprises at least one short-chain polyol.

27. The composition according to claim 26, wherein the at least one polyol is present in an amount ranging from less than 5% by weight relative to the total weight of the composition.

28. The composition according to claim 26, wherein the at least one polyol is chosen from glycerol and propylene glycol, and mixtures thereof.

29. The composition according to claim 1, wherein the composition further comprises at least one additive chosen from:
conditioning cationic polymers,
humectants,
emollients,
fillers,
refreshing agents and calmatives,
lubricants,
vitamins,
dyes,
fragrances,
antioxidants,
antibacterial agents and/or antifungal agents, and
preserving agents.

30. A shaving process for skin comprising:
applying to the surface of the skin to be shaved a soap-free shaving composition, packaged in a monoblock aerosol device and producing a foam at the outlet of said device, said composition comprising, in a cosmetically acceptable medium:
a) at least one aqueous phase;
b) at least one N-acylsarcosine, the acyl radical of which is C₁₀-C₂₀ and which is from 50% to 90% neutralized;
c) at least one non-neutralized saturated linear C₁₄-C₁₈ fatty acid;
d) at least one nonionic surfactant; and
e) at least one propellant, followed by shaving the hairs using a razor.

31. A shaving kit comprising:
a) at least one soap-free shaving composition, packaged in a monoblock aerosol device and producing a foam at the outlet of said device, said composition comprising, in a cosmetically acceptable medium:
1) at least one aqueous phase;
2) at least one N-acylsarcosine, the acyl radical of which is C₁₀-C₂₀ and which is from 50% to 90% neutralized;
3) at least one non-neutralized saturated linear C₁₄-C₁₈ fatty acid;
4) at least one nonionic surfactant; and
5) at least one propellant,
b) at least one razor, and/or
c) a means for spreading a shaving composition.

32. The shaving kit according to claim 31, wherein the shaving kit further comprises an aftershave composition for soothing razor burn.
